(19) United States
(12) Patent Application Publication
(10) Pub. No.: US 2010/0212406 A1
(43) Pub. Date: Aug. 26, 2010

(54) COLLECTION OF DISSOLVED GASES FROM GROUNDWATER

(76) Inventors: Bryant A. Browne, Stevens Point, WI (US); Kathryn Lane Browne, Stevens Point, WI (US)

Correspondence Address: PATTERSON THUENTE CHRISTENSEN PEDERSEN, P.A., 4800 IDS CENTER, 80 SOUTH 8TH STREET, MINNEAPOLIS, MN, 55402-2100, US

(21) Appl. No.: 12/392,572

(22) Filed: Feb. 25, 2009

(51) Int. Cl. G01N 33/18; B01D 19/00; B01D 35/06; B01D 35/157; B01D 35/26; B01D 35/01

(52) U.S. Cl. 73/61.41; 96/194; 96/16; 95/22

(57) ABSTRACT

A system for rapid collection of large volumes of dissolved gases from groundwater pumps the water from a large-diameter passage through a small-diameter restrictor passage, and then into a flow-through collection chamber. The small-diameter passage causes a drop in the hydrostatic pressure of the groundwater traveling therein, causing spontaneous ebullition of gas bubbles, which are then collected in the collection chamber. A treatment station within the passages selectively alters the concentration of dissolved gases within the groundwater. Testing reveals that the gases are collected in proportion to their presence in the groundwater, and thus the system allows accurate quantification of concentrations of dissolved gas in groundwater. The system may beneficially be made easily portable, thereby allowing its use in the field, as well as in laboratory settings.

### CROSS-REFERENCE TO RELATED APPLICATIONS, IF ANY

The application claims the benefit of the filing date of U.S. Provisional Patent Application No. 60/903,631, filed on Feb. 25, 2008.

### STATEMENT REGARDING FEDERALLY SPONSORED RESEARCH OR DEVELOPMENT

Not applicable.

### REFERENCE TO A MICROFICH APPENDIX, IF ANY

Not applicable.

### BACKGROUND OF THE INVENTION

1. Field of the Invention

The present invention relates generally to methods and apparata for collecting and analyzing gases dissolved within liquids, and, more specifically, to the collection and analysis of dissolved gas within groundwater, and, most specifically, to methods and apparata for analyzing carbonaceous species present within groundwater.

2. Background Information

Groundwater, i.e., subsurface waters such as aquifers, wells, etc. and surface waters such as lakes, streams, etc., is a frequent subject of environmental and biogeochemical study. Often, the characteristics and behavior of the groundwater can be at least partially determined by studying gases dissolved within the groundwater. As examples, dissolved gases are often used to generate estimates of denitrification from excess N₂; to study greenhouse gases in groundwater; to evaluate terminal electron accepting processes using H₂; and to study the cycling of biogeochemically important trace gases such as CO₂, CH₄, and N₂O. Additionally, groundwater studies often utilize dissolved gases as “tracers” which allow tracking of environmental processes, with applications including paleothermometry, age-dating, estimating groundwater recharge temperature, measuring advection and dispersion in rivers and streams, tracing of ocean mixing and circulation paths, and tracking of volatile pollutants in groundwater, among other applications.

However, the measurement of dissolved gases can be a major analytical challenge. For example, when atmospheric gas concentrations are abundant relative to the dissolved gas concentrations, contamination during sampling and analysis is a major concern. Losses during handling and storage can also be a major problem for some gases due to their volatility and/or biodegradability. In addition, for gases that are typically present at very small concentrations (e.g., SF₆ and noble gas isotopes), simply attaining a large enough sample to generate a measurable signal can be a major hurdle.

In view of the foregoing difficulties and the importance of obtaining accurate dissolved gas measurements, a wide variety of sampling and storage procedures have evolved for dissolved gases. Groundwater sampling methods include collection of water in sealed bottles (with or without chemical preservation) for equilibration of gases in the head space above the water sample, flame sealing of water samples in glass ampules under high purity gases to protect the sample from contamination with the atmosphere, collection in copper tubes with stainless steel pinch-offs, bubble stripping, diffusion probes, and downhole samplers, and others. Complex devices and methods may also be required to extract the gases from the groundwater and to introduce and process gas samples within an analytical instrument. These include, for example, purge and trap devices for analysis of volatile organic compounds (VOCs), trace gases, chlorofluorocarbons (CFCs) and SF₆, and highly refined vacuum extraction devices in-line with analytical instruments.

The forgoing devices and methods for groundwater sampling and dissolved gas collection suffer from the problems that can be time-consuming, expensive, and difficult to operate, particularly in field conditions. Thus, it would be useful to have available further devices and methods for groundwater sampling and dissolved gas collection and analyses which at least partially overcome some of these difficulties.

In response to the above-mentioned difficulties, applicant previously disclosed a novel method and novel apparatus for collecting dissolved gases from ground water employing pumping-induced ebullition. The invention was disclosed in U.S. Pat. No. 6,706,094, which issued Mar. 16, 2004. The novel method and apparatus disclosed in this U.S. Patent are hereby incorporated by reference in their entireties.

Applicant has now devise a method and apparatus for pretreating ground water followed by collecting dissolved gases from the treated ground water employing pumping-induced ebullition.

### SUMMARY OF THE INVENTION

The invention involves methods and apparata for dissolved gas collection which are intended to at least partially solve the aforementioned problems. To give the reader a basic understanding of some of the advantageous features of the invention, following is a brief summary of embodiments of the collection devices and methods. As this is merely a summary, it should be understood that more details regarding the embodiments may be found in the Detailed Description set forth elsewhere in this document. The claims set forth at the end of this document then define the various embodiments of the invention in which exclusive rights are secured.

An apparatus for collecting dissolved gases from groundwater is provided wherein the groundwater of interest for study is supplied to an intake line having an inlet end and a downstream outlet end. One or more restrictor lines are then provided downstream from the outlet end of the intake line, with the restrictor lines promoting a hydrostatic pressure drop in the groundwater traveling therein. In one embodiment of the apparatus, the intake line and restrictor lines are one and the same. This pressure drop can be generated owing to pumping forces working against resistance (i.e., frictional losses) from the restrictor lines, which resistance may be enhanced by decreasing the flow area of each restrictor line (i.e., the area of the flow passage measured perpendicular to the direction of flow) and/or increasing the length of each restrictor line. Alternatively or additionally, a pressure drop can be generated or enhanced by having the combined flow areas of the restrictor lines be less than the flow area of the intake line, whereby the fluid velocity of the groundwater is increased to generate a corresponding pressure drop. In the present process, it is imperative that the pumping rate is sufficient to provide an increase in fluid velocity in the restrictor line(s) to generate the pressure drop therein. When the groundwater is supplied from the intake line and its pressure is decreased within the restrictor lines, the dissolved gases (if any) within the groundwater will precipitate from the groundwater. Thus the dissolved gases begin to bubble from the groundwater flowing within the restrictor lines.

The intake line also includes at least one treatment station positioned therein, with the at least one treatment station providing selected treatment of fluid flow through the intake line. In one embodiment wherein the intake line and restrictor line(s) are one and in the same, the at least one treatment station is positioned within the restrictor line(s). If effect, the at least one treatment station is position upstream of a pump that draws groundwater through the apparatus. The treatment station can include at least one inlet mixing T within the intake line, the at least one inlet mixing T introducing a treatment fluid into the fluid flow through the intake line. The treatment station can include an electromagnetic radiation source within the intake line, positioned downstream of the at least on inlet mixing T, the electromagnetic radiation source exposing fluid flow through the intake line to electromagnetic radiation. Multiple mixing Ts may be present, alone or in combination with a downstream electromagnetic radiation source. The at least one treatment station functions to selectively alter the concentration of any gases dissolved in the ground water.

A gas collection chamber is then situated downstream from the restrictor line(s), with the gas collection chamber having an interior wherein the precipitated gas bubbles may collect near its upper side. Thus, as the flow of groundwater continues, the amount of collected gas at the upper side of the gas collection chamber grows. A disposal line then exits the gas collection chamber, for example, near the lower side of its interior and below the level at which the groundwater from the restrictor lines enters the gas collection chamber, so that degassed groundwater will flow from the disposal line but the collected gases remain within the gas collection chamber.

IF the source of the groundwater is not pressurized such that it will flow through the lines of the apparatus of its own accord (as where the groundwater is supplied from an artesian well), a pump is situated between the foregoing components to induce flow. In one embodiment, a pump is positioned downstream from the restrictor lines and upstream of the gas collection chamber, so that the pressure drop within the restrictor lines (and thus the precipitation of any gases from the groundwater within the restrictor lines) is enhanced by the pump suction. In practice, the apparatus is configured with a pump device positioned downstream from the restrictor line(s) and upstream of the gas collection chamber, providing optimum operation of the apparatus.

To remove at least a portion of the collected gases from the gas collection chamber, a sampling port can be provided at or near the upper side of the interior of the gas collection chamber where the precipitated gases collect. The sampling port preferably bears a valve, and is adapted for removable attachment of a sample collector (such as a syringe) having an adjustable interior volume. Thus, the valve may be opened and the collected gases may be drawn off into the sample collector to be provided to a suitable analysis device. The collected gases may then be analyzed by gas chromatography or other methods to determine their contents. Alternatively, analytical instrumentation for gas analysis can be directly connected to the sampling port, via a bypass valve, so that analysis can occur concurrently with collection of gases.

Conveniently, the foregoing apparatus may be provided in a portable and easily disassembled and reassembled form so that it can be easily used in the field as well as in a laboratory. As an example, the intake and restrictor lines may be provided by easily folded flexible plastic tubing, and the collection chamber, which is preferably formed of rigid plastic or glass, can be sized so that it may be easily held by one hand. The pump, if present, can take the form of a laboratory peristaltic (or other) pump which has an input shaft adapted for receiving a rotary input from a cordless drill or other easily portable source of a rotary power input. Alternatively, a gear pump magnetically coupled to a rotary power source is employed to pump groundwater through the apparatus. The use of other suitable pumping devices is also contemplated. Some of all of the foregoing components can be transparent, allowing the color and particulate content of the sample groundwater to be observed during gas collection, and allowing any areas of actual or potential fouling to be observed during gas collection.

Embodiments of the invention offer several advantages for gas collection and analysis applications. Initially it allows multiple sample collection procedures to be combined into one single method. To illustrate, traditional sampling techniques might require
(a) that water samples be analyzed for CFCs are flame sealed in glass ampules under high purity N₂, with subsequent analysis by purge and cryogenic trap.
(b) that water sample to be analyzed for SF₆ are collected in a one-liter or larger bottle with a special cap, with subsequent analysis by purge and cryogenic trap,
(c) that water samples to be analyzed for Ar, N₂, and O₂ (which is unstable during storage) are collected in a 50-ml septum-sealed glass bottle, with subsequent analysis by a head space method,
(d) that water samples to be analyzed for H₂, with subsequent analysis by direct injection, and/or
(e) that water to be analyzed for CH₄, and N₂O be injected into a He or Ar flushed septum-sealed glass bottle, with subsequent analysis of the CH₄ and N₂O in the He or Ar head space.

In contrast, by use of the invention, one only need collect one or more samples of the collected gas in the field or elsewhere, using a syringe or other sample collector, for later direct injection into a gas chromatograph in the laboratory, through purge and trap and/or other methods can be used if desired. Alternatively, the collected gases can be continuously supplied to analytical instrumentation concurrently with their collection in the field or in the laboratory.

Further, embodiments of the invention avoid the consumption and production of biogenic gases that can occur during storage of a water sample for later analysis. Biotransformation of gases such as CO₂, O₂, CH₄, and N₂O during storage of water sample causes over- or underestimation of the in situ concentration of such gases. Since the invention separates the gases from the water when the water is sampled, only the gas need be stored, thus avoiding aqueous bio-transformations during storage.

Additionally, embodiments of the invention allow the collected gases to be directly supplied to or injected into analytical instrumentation in the field or in the laboratory. More elaborate sample introduction and processing approaches (e.g., the valving required to purge and trap CFCs and SF₆ from water samples) can be unnecessary depending on the application in question.

Further advantages, features, and objects of the invention will be apparent from the following detailed description of the invention in conjunction with the associated drawings.

### BRIEF DESCRIPTION OF THE DRAWINGS

FIG. 1 depicts a simplified diagram of one embodiment of an exemplary version of the apparatus of the present invention.

FIG. 2 depicts a simplified diagram of another embodiment of an exemplary version of the apparatus of the present invention.

### DESCRIPTION OF THE EMBODIMENTS

### Nomenclature

10 Groundwater

100 PIE Apparatus

102 Intake Line

104 Inlet End

106 Outlet End

108 Inlet Ends

110 Restrictor Lines

112 T-Connector

114 Outlet Ends

116 Inlet End

118 Output Line

120 Pump

122 Input Shaft

124 Output End

126 Collection Chamber

128 Interior Chamber

130 Lower Side of Chamber

132 Disposal Line

134 Upper Side of Chamber

136 Sampling Port

138 Valve

140 Sample Collector

142 Mouth of Sample Collector

144 Interior of Sample Collector

146 Plunger

150 Treatment Station

152 Inlet Mixing T

154 Fluid Reservoir Container

156 Transfer Line

158 Control Valve

160 Electromagnetic Radiation Source

162 Electrical Conductors

200 PIE Apparatus

238 Three-Position Valve

240 Water Outlet Line

242 Gas Outlet Line

244 Detector Cell

### Construction

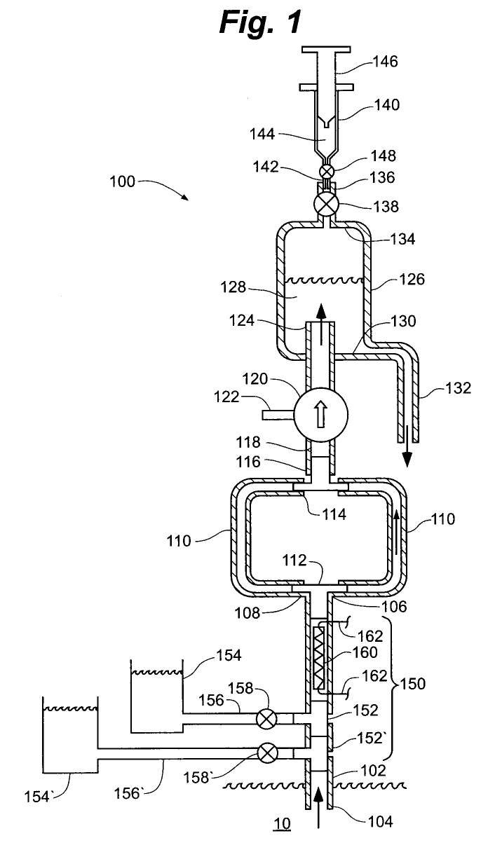

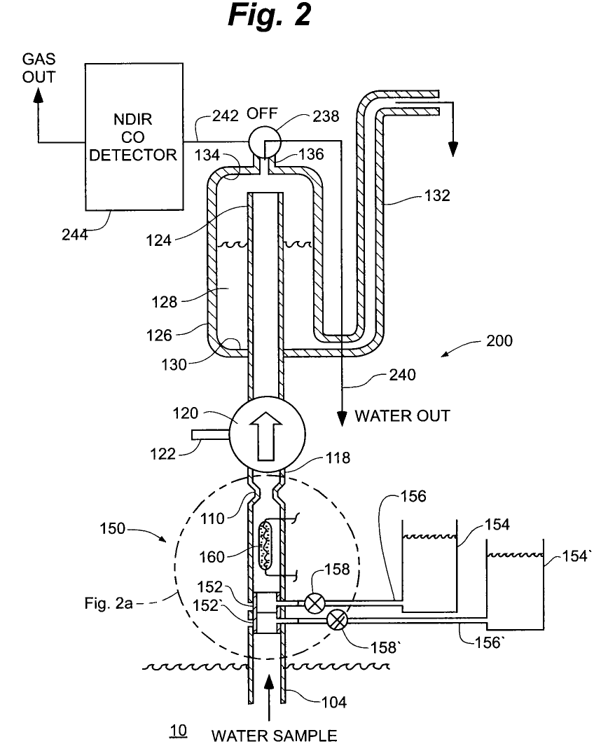

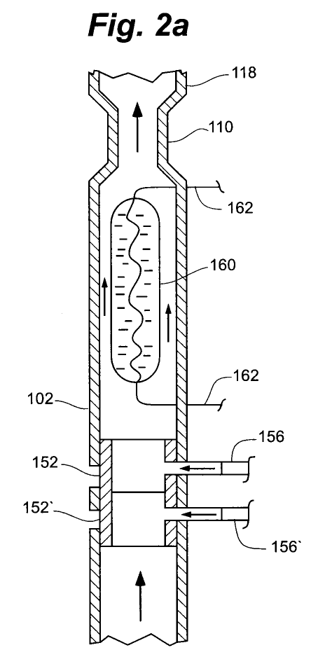

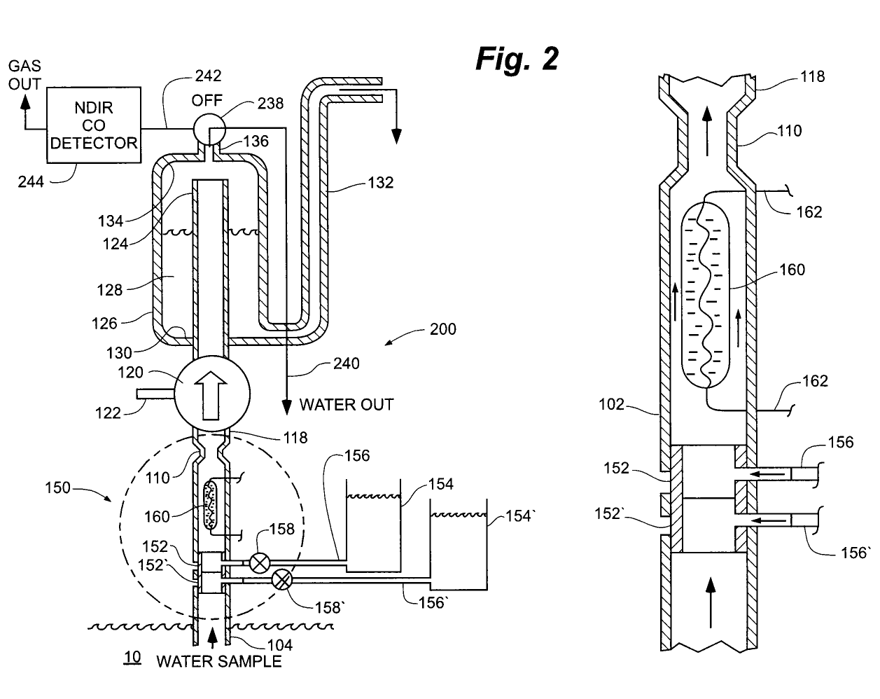

FIG. 1 illustrates on exemplary embodiment of the apparatus of the invention at 100. An intake line 102 has its inlet end 104 situated within a source of groundwater 10, and its outlet end 106 is connected in fluid communication with inlet ends 108 of a pair of restrictor lines 110 via a T-connector 112. It should be understood that the intake line 102, the restrictor lines 110, and other components are not shown to scale in FIGS. 1 and 2, nor are they proportionately illustrated, and in reality they will generally have significantly greater length than the apparent length depicted in the Figures. The outlet end 114 of the restrictor lines 110 are similarly connected in fluid communication with the inlet end 116 of an output line 118. In an alternative embodiment of the apparatus, the intake line and restrictor line(s) are one in the same. A pump 120 is situated in the output line 118 to provide the driving force for supplying the groundwater through the apparatus 100, and the pump 120 can be provided in a portable form with a protruding input shaft 122 which is adapted for removable attachment to a separate driver, such as a cordless drill, which can supply a rotary input to the input shaft 122. Alternatively, a gear pump magnetically coupled to a rotary power source is employed to pump groundwater through the apparatus 100. The use of other suitable pumping devices is also contemplated. The outlet end 124 of the output line 118 can be provided within the interior 128 of the collection chamber 126 above its lower side 130 so that a disposal line 132 leading from the collection chamber 126 at or near its lower side 130 can drain off water from the interior 128 of the collection chamber 126, while any gas bubbles exiting from the outlet end 124 of the output line 118 can float toward the upper side 134 of the collection chamber 126 for collection. Thus, by having the groundwater flow from the restrictor lines 110 enter the collection chamber 126, gas bubbles can be collected without their disposal through the disposal line 132 along with excess groundwater.

The intake line 102 also includes at least one treatment station 150 positioned therein, with the at least one treatment station 150 providing selected treatment of fluid flow through the intake line 102. In an embodiment wherein the intake line 102 and restrictor line(s) 110 are one in the same, the at least one treatment station 150 is positioned within the restrictor line(s) 110. If effect, the at least one treatment station 150 is positioned upstream of the pump 120 that draws groundwater through the apparatus 100. The treatment station 150 can include at least one inlet mixing T 152 within the intake line 102, the at least one inlet mixing T 152 introducing a treatment fluid into the fluid flow through the intake line 102. For example, an inlet mixing T 152 is connected to a fluid reservoir container 154 via a transfer line 156 that is under the control of a valve 158. The inlet mixing T 152 introduces a liquid reagent into the fluid flowing within the intake line 102 to alter the constituents of the groundwater therein. In practice, the use of a small internal diameter transfer line 156 delivers highly reliable volumes of treatment fluid to the inlet mixing T 152.

Additionally, the treatment station 150 can include an electromagnetic radiation source 160 within the intake line 102, positioned downstream of the at least one inlet mixing T 152, the electromagnetic radiation source 160 exposing fluid flow through the intake line 102 to electromagnetic radiation to alter the constituents of the ground water therein. The electromagnetic radiation source 160, such as an ultraviolet lamp, occupies a portion of the internal cross section of the intake line 102, thereby minimizing the thickness of the groundwater passing between the ultraviolet lamp and the wall of the intake line 102. The electromagnetic radiation source 160 is connected to a power source (not shown) exterior the intake line 102 by electrical conductors 162 that transverse the wall of the intake line. Alternatively, the electromagnetic radiation source 160 is configured similar to a laboratory reflux condenser, with the bulb of the electromagnetic radiation source 160 forming the bore of the condenser, and the groundwater flowing through the cooling jacket surrounding the bulb. Multiple inlet mixing Ts 152 can me present, alone or in combination with a downstream electromagnetic radiation source 160, as illustrated in the Figures. The at least one treatment station 150 functions to selectively alter the concentration of any gases dissolved in the groundwater.

When the pump 120 is actuated to achieve a sufficiently high flow rate, the aforementioned gas bubbles form in the restrictor 110. It is believed that this occurs owing to the following principles. For a bubble to form in water, the sum of partial pressures (ΣPᵢ) of volatile gas species (N₂, O₂, Ar, etc.) must be in excess of the ambient pressure PH.

ΣPᵢ=PN₂+PO₂+Par+PH₂)+ . . . >PH

Thus, spontaneous formation of bubbles (ebullition) can be induced by decreasing the hydrostatic pressure on the right-hand side of the equation. In the apparatus 100, wherein the pump 120 is situated downstream from the restrictor lines 110, the suction from the pump 120 works on the fluid restrictor lines 110. The restrictor lines 110 offer relatively high resistance to this suction owing to fluid friction within the restrictor lines 102, which may be enhanced by making the flow areas of the restrictor lines 110 smaller and/or by making the restrictor lines 110 longer. Thus, the fluid within each of the restrictor lines 110 is subjected to a drop in hydrostatic pressure upstream from the inlet of the pump 120, causing bubble generation to occur in these areas. The theoretical basis for the invention's operation may be explained by the Darcy-Weisbach equation, which may be expressed as:

hf=(f/2g)(V²/D)L=(16/π²)(f/2g)(Q²/D⁵)L

Where hf is the head loss in a fluid passage;

f is the resistance coefficient;

g is the gravitational constant;

L is the length of the fluid passage;

D is the diameter of the passage;

V is the mean velocity of the fluid in the passage; and

Q is the volumetric flow rate of the fluid in the passage.

Thus, it is seen that the head loss is dependent on (1) the diameter of the slow passage; (2) the velocity/flow rate of the fluid; and (3) the length of the fluid passage.

Bubble generation and gas collection can also be enhanced if additional or alternative measures are taken to enhance the pressure drop in the restrictor lines 110. Initially, the restrictor lines 110 can be provided with a lower combined flow area that the flow area of the intake line 102. If the flow area along a fluid passage is decreased, conservation of mass dictates that the fluid must necessarily undergo a velocity increase in the decreased flow area. Pressure will then decrease in the decreased flow area owing the velocity increase experienced by the fluid (with increased velocity resulting in the pressure decrease as per Bernoulli's equation). In the embodiment wherein the intake line 102 and the restrictor line(s) 110 are one in the same, the groundwater source is drawn directly in the restrictor line(s) 110. A decrease in hydrostatic pressure within the restrictor lines 110 may also be promoted where the restrictor lines 110 are situated at a greater height than the groundwater source 10, since fluid necessarily undergoes a decrease in hydrostatic pressure as it rises through a column.

It should be understood that more or less than two restrictor lines 102 may be used, with the foregoing design considerations being taking into account. More than one restrictor line 110 will offer more than one flow area wherein bubbles may be generated, thus potentially allowing an increase in the rate of gas collection within the collection chamber 126.

The pump 120, while depicted directly downstream from the restrictor lines 110, may be situated elsewhere within the fluid circuit (e.g. within the disposal line 132). While the pump 120 repressurizes the two-phase gas bubble/groundwater mixture when it is situated between the restrictor lines 110 and the gas collection chamber 126, it has been found that such repressurization is of sufficiently short duration that the gas bubbles do not significantly redissolve within the groundwater prior to entering the collection chamber 126, and thus the collected gas yield is not significantly diminished. The pump 120, if not self-priming, is easier to prime when it is situated nearer the inlet end 104 of the intake line 102, but since the inlet end 104 can be submerged in a stream or pound, or inserted within a borehole, the versatility of the apparatus 100 can be diminished if the pump 120 is situated too near the inlet end 104.

The collection chamber 126 is then situated downstream from restrictor lines 110 wherein the gas bubbles precipitate. Sufficient overhead space is provided in the collection chamber 126 that gas bubbles will buoyantly float to the upper side 134 of the collection chamber 126, allowing the gas bubbles to be harvested for analysis. Since gas partial pressures may be presumed to be proportional to gas mole fractions as per Dalton's Law, the partial pressures of individual gases within the collection chamber 126 can be determined, and used to quantify the gas content in the bulk fluid.

To harvest the gases collected at the upper side 134 of the collection chamber 126, the collection chamber 126 can be provided with a sampling port 136, and a valve 138 which selectably allows access of the interior 128 of the collection chamber 126. A sample collector 140 can be provided which has a mouth 142 which is removably attachable to the sampling port 136, and which has an interior 144 with adjustable size. Thus, when the sample collector mouth 142 is fit on the sampling port 136, the valve 138 may be opened, and the ample collector interior 144 can be expanded to draw off the gases collected within the collection chamber 126 into the sample collector 144. Here the sample collector 140 is depicted as a syringe wherein its interior 144 can be expanded when the plunger 146 is withdrawn, though other forms of sample collectors are possible. In practice, it is found that collection of gases from the collection chamber 126, via the sampling port 136, into evacuated septum bottles, provides samples that are stable to storage for greater than six (6) weeks. A valve 148 can also be provided on the sample collector 140 so that any gases withdrawn into its interior 144 may be sealed therein to allow transport of the gases to a gas chromatograph or other analysis device. A sample collector could instead take the form of a sample chamber which can be removably connected to the collection chamber 126 and selectively closed at its inlet, as with the syringe 140, but which does not have an interior with adjustable size, such as the evacuated septum bottles referred to above. Instead, the ability to expel the collected gas from the interior of the sample chamber might be provided by having a separate second inlet through which a carrier gas or displacing fluid might be introduced to allow displacement of the collected gas from the sample collector's interior.

The apparatus 100 is preferably designed as a compact unit which can be readily carried into the field for gas sampling at remote locations, and thus it is useful to provide at least the intake line 102 and restrictor lines 110 in the form of flexible tubing which may be readily coiled and assembled/disassembled when desired. The collection chamber 126 can be rigid for ease of handling. The pump 120, which will also generally be rigid, can be positioned adjacent the collection chamber 126 so that all rigid components are adjacently situated for easier storage and handling allowing the intake line 102 and restrictor lines 110 to simply be coiled about the rigid components from transport and uncoiled for use. Most or all of the various components can be formed of transparent materials (e.g., to form the intake 102 and restrictor lines 110 of the transparent tubing, and the collection chamber 126 of the transparent materials) so that a user can monitor the apparatus 100 for fouling during its use, and/or bubble formation within the restrictor lines 110 and gas collection within the collection chamber 126 cab be monitored.

Embodiments of the apparatus 100 have been tested using transparent using transparent nylon tubing (2.0 mm inner diameter) as the intake line 102; two 3-meter segments of nylon tubing (2.0 mm inner diameter) as the restrictor lines 110; an output line 118 formed of 0.8 m of the same nylon tubing (2.0 mm inner diameter); a self-priming masterflex Model U 07024-21 tubing pump head (Cole-Parmer Instrument Co., Vernon Hills, Ill.) for pump 120; and a standard laboratory counterflow trap (made of glass) for the collection chamber 126. When the pump 120 was driven at approximately 1000 rpm by a cordless drill, which is a particularly convenient and portable pump driver for field use, the pressure in the restrictor lines 120 upstream from the pump 120 decreased to an estimated 0.1 atm, and a groundwater throughput of approximately 340 ml/min was achieved. Alternatively, a gear pump magnetically coupled to a rotary power source is employed to pump groundwater through the apparatus 100. The use of other suitable pumping devices is also contemplated. The output line 118 extended into the collection chamber 126 to provided approximately 5 cm of head space adjacent the upper side 134 of the collection chamber 126 wherein gases could collect.

An exemplary gas collection procedure is as follows. Before sample collection, the disposal line 132 is raised above the collection chamber 126 to establish a back pressure against which the pump 120 will work. About one liter of groundwater is then pumped with the sampling port valve 138 opened, forcing groundwater flow through both the sampling port valve 138 and the disposal line 132, cleaning the collection chamber 126 and displacing all gases therein. The sampling port valve 138 is then closed, leaving only water within the collection chamber 126. As water is pumped to the disposal line 132, gas collection occurs at the upper side 134 of the collection chamber 126. When sufficient gas has accumulated, pumping is stopped and the sampling port valve 138 and sample collector valve 148 are opened to allow collection of the gases into the gas-tight sample collector 140. If the disposal line 132 is raised so that its water level is held even with the water level at the upper side 134 of the collection chamber 126, the pressure within the collection chamber 126 and sample collector 140 are approximately the same as ambient atmospheric pressure during collection of gases into the sample collector 140.

When the foregoing arrangement is used with standard groundwater sources, approximately 5 to 8 ml of gas (at approximately atmospheric pressure) per liter of water may be collected at a pumping rate of about 340 ml/min. The total amount of gas collected depends on how long the sampling period lasts. So long as the groundwater supply is not limited, embodiments of the invention allow collection of a sufficiently large volume of gas that trace gases, such as CFCs and SF₆ can be accumulated in sufficient amounts that their content and amount may be amore accurately measured.

The collected gas can then be analyzed using a variety of known methods, with an exemplary analysis method proceeding as follows. Gas samples collected from groundwater were maintained in gas-tight 10 ml syringes until analysis. Water temperature and ambient barometric pressure were measured in the filed during extraction of the gas. The total pressure P_{T}, of dissolved gas was measured relative to laboratory barometric pressure BP_{lab} using a total dissolved gas saturation monitor in percent saturation mode:

P,=BP,a,−% SA T/100

The gas samples were then analyzed for general content. N₂, Ar. O₂, N₂O, CO₂, and H₂ were detected using a pulse discharge detector (PDD) in the helium ionization mode, SF₆ was detected using a PDD in electron capture mode, and chlorofluorocarbons were detected using a ⁶³Ni electron capture detector. Gas chromatography was then used to measure the mole fractions (Xᵢ) of the detected gases (.N₂, Ar. O₂, N₂O, CO₂, CH₄, H₂, CFC11, CFC12, CFC13 and SF₆). The chromatographic conditions for each of the gaseous components are detailed in U.S. Pat. No. 6,706,094, incorporated herein by reference in its entirety. Conditions were modified (e.g., substitution of He carrier gas) as necessary to accommodate use of a PDD. The samples, along with gas standards, were transferred from syringes to a sample loop on a six-port valve for injection onto the GC column. Six point calibration curves (mole fraction versus peak area) were performed by injection of gas dilutions prepared by mixing precisely measured aliquots of blank gases and standard gas. In load position, the sample loop was maintained at ambient lab temperature and pressure prior to injection.

After gas chromatography yielded mole fractions, the partial pressure PI of each gas in the water sample was calculated using the following relationship:

Pᵢ=Xᵢ·P_{T}/F_{c,i}

Here the product Xᵢ·P_{T} equals the partial pressure of the gas in the water sample, and F_{c,i} is a gas specific fractionation coefficient which accounts for gas solubility and other factors:

F_{c,i}=X_{i,M}/X_{i,A}

where X_{i,M} is the measured mole fraction of a reference sample of the gas in question when this gas is extracted from equilibrated water by use of the invention, and XI, is the mole fraction of the same gas as measured from direct injection of a reference sample into a chromatograph. Because embodiments of the invention to some degree forcibly extracts gases from water, and owing to the solubility of each gas in water (among other factors), the amounts of the extracted gases might differ from those that would occur under equilibrium conditions (i.e., if the gases were allowed time to come to equilibrium in a head space above water). It was found that for most gases, the variance of the fractionation coefficient F_{c,i} from unity was negligible or small (i.e., differences between X_{i,M} and X_{i,A} are negligible or small). However, some gases had marked differences in extractability between the amounts recovered by a method of the invention and the amounts recovered at equilibrium. For example, SF₆ (which has low solubility in water) had a fractionation coefficient F_{c,i} near 0.5. In contrast, CFC11 (higher solubility) had a fractionation coefficient F_{c,i} near 1.2. However, the fractionation coefficients F_{c,i} for different gases did not always correspond to their solubility, suggesting that the basis for deviation of fractionation coefficients F_{c,i} from unity requires further investigation.

The individual gas concentrations determined by use of the invention were checked for accuracy versus results obtained by other methods and versus known concentrations in reference gas mixtures, and it was found that the invention allows accurate extraction of gases from groundwater and measurement of their concentrations. The measured gas concentrations compared favorably to concentrations measured by other methods, e.g., head space equilibration, membrane electrode and Winkler titration methods (for O₂), etc.

Depending on the quality of the syringes or other sample collectors used, there may be limitations on the duration of sample integrity. Thus, samples are best analyzed as soon as possible after they are collected. However, because the invention reduces sample preparation in the lab, analysis speeds can effectively be increased, thereby reducing any backlog of specimens waiting to be tested and allowing newly-obtained samples to be analyzed sooner. In practice, it is found that collection of gases from the collection chamber 126, via the sampling port 136, into evacuated septum bottles, provides samples that are stable to storage for greater than six (6) weeks.

Referring now to FIG. 2, an alternative embodiment of the apparatus 200 of the invention is illustrated. The portion of the apparatus 100 (FIG. 1) that is upstream of the collection chamber 126 is essentially the same as shown in FIG. 1, and need not be described in detail. In the embodiment wherein the intake line 102 and restrictor line(s) 110 are one, the at least one treatment station 150 is positioned within the restrictor line(s) 110. If effect, the at least one treatment station 150 is positioned upstream of the pump 120 that draws groundwater through the apparatus 200. In FIG. 2, a single restrictor line 110 is shown for simplicity. The disposal line 132 is configured such that the flow of ground water exits the collection chamber 126 at a level sufficient to provide a slight positive pressure on gases collecting within the collection chamber 126.

In this embodiment, the sampling port 136 of the collection chamber 126 includes a three-position valve 238. The three-position valve 238 is rotatable to selectively provide the functions of water-out, off, or gas-out. Two outlet lines are connected to the three-position valve 238, a water outlet line 240 (for the water-out function) and a gas outlet line 242 (for the gas-out function). The water outlet line 240 allows for purging the collection chamber 126 of gases, the off position allows gas collection in the collection chamber 126, and the gas outlet line 242 allows for transfer of collected gases to the detector cell 244 for analysis. For example, the detector cell 244 may be a nondespersive infrared detector (NDIR) used to selectively detect CO, as illustrated in FIG. 2. The NDIR detector cell 244 is configured as a flow through cell for continuous monitoring of CO₂.

The configuration of the apparatus 200 illustrated in FIG. 2 finds particular use in determining the distribution of carbonaceous species in fluids, such as ground waters. Carbonaceous species in ground water can be present as dissolved CO₂, inorganic carbon (IC), including dissolved CO₂, bicarbonate and carbonate (HCO₃⁻, CO₃^{═}), and organic carbon (OC). Employing a combination of treatment stations 150 in the inlet line 102, the distribution of carbonaceous species in a groundwater 10 is readily determined.

In mode I, the ground water passing through the apparatus 200 receives no treatment when drawn through the treatment stations 150. In this mode, only dissolved phase and the concentration of CO₂, denoted as “A,” determined using the NDIR detector cell 244.

In mode II, the ground water passing through the apparatus 200 receives continuous acidification at treatment stations 150. A concentrated solution of a strong acid, such as sulfuric acid or phosphoric acid, is added via one inlet mixing T 152. The acid solution is maintained in the reservoir container 154 and supplied to the inlet mixing T 152, under the control of a valve 158, through the supply line 156. In practice, the use of a small internal diameter transfer line 156 delivers highly reliable volumes of treatment fluid to the inlet mixing T 152. Acidification treatment of the ground water converts all bicarbonate and carbonate (HCO₃⁻, CO₃^{═}) to dissolved CO₂. The resulting total dissolved CO₂, designated as inorganic carbon (IC), is then liberated into the gas phase and the total concentration of CO₂, denoted as “A+B,” determined using the NDIR detector cell 244. Thus, the bicarbonate and carbonate (HCO₃⁻, CO₃^{═}) content “B” of the ground water is determined as the value (A+B)−(A)=B.

In mode III, the ground water passing through the apparatus 200 receives multiple treatments at treatment stations 150. In addition to continuous acidification at treatment stations 150, as described above, a concentrated solution of an oxidizing agent, such as potassium persulfate (K₂S₂O₈), is added via a second inlet mixing T 152′. The oxidizing agent solution is maintained in the reservoir container 154′ and supplied to the inlet mixing T 152′, under the control of a valve 158′, through the supply line 156′. In practice, the use of small internal diameter transfer lines 156, 156′ deliver highly reliable volumes of treatment fluid to the inlet mixing Ts 152, 152′. The acidified ground water containing oxidizing agent then passes through the electromagnetic radiation treatment station 160 to oxidize all organic carbon (OC) species to dissolved CO₂. The electromagnetic radiation treatment station 150 includes an ultraviolet light source 160 for irradiating the acidic ground water containing oxidizing agent as the ground water passes around the ultraviolet light source 160, thereby oxidizing all organic carbon-containing compounds to CO₂. The resulting total dissolved CO₂ is then liberated into the gas phase and the total concentration of CO₂, denoted as “A+B+C,” determined using the NDIR detector cell 244. Thus, the organic carbon (OC) content “C” of the ground water is determined as the value (A+B+C)−(A+B)=C.

The above described application of the apparatus 200 for carbonaceous component determination in ground water is but one functional aspect of the present invention. Other applications combining at least one treatment station with the PIE system 200 are contemplated.

The apparata 100 and 200 of the invention need not take the form of a portable apparatus, and may instead take the form of a semi-permanent or permanent installation if desired (e.g., near landfills or other sites where groundwater monitoring is useful). It is not necessary that the collected gases necessarily be analyzed; for example, where groundwater contamination is present, the embodiments of invention might be used to decrease the pressure of the groundwater so that any volatile contaminants flash off into gas for collection, and the separated groundwater may be allowed to flow back to its source.

It is understood that embodiments of the invention have been described above in order to illustrate how to make and use the invention. The invention is not intended to be limited to these embodiments, but rather is intended to be limited only by the claims set out below. Thus, the invention encompasses all different embodiments that fall literally or equivalently within the scope of these claims.

What is claimed is:

1. An apparatus for collecting dissolved gases from groundwater comprising:
an intake line having an intake line inlet end and a downstream outlet end, the intake line having an intake line flow area defined therein;
at least one restrictor line extending between an restrictor line inlet end and a downstream outlet end, the at least one restrictor line having a flow area defined therein, wherein the restrictor line inlet end of the at least one restrictor line is in fluid communication with the intake line;
a pump positioned downstream from the at least one restrictor line;
at least one treatment station positioned upstream of the pump, the at least one treatment station adapted for selected treatment of fluid flow through the intake line;
a gas collection chamber positioned downstream from the pump, the gas collection chamber having an upper side, an opposing lower side, and an interior; and
a disposal line exiting the gas collection chamber.

2. The apparatus of claim 1, wherein any fluid flow from the outlet end of the at least one restrictor line is received within the interior of the gas collection chamber above a height at which the disposal line exits the gas collection chamber.

3. The apparatus of claim 1, wherein the disposal line exits the gas collection chamber adjacent the lower side of the gas collection chamber.

4. The apparatus of claim 1 further comprising a sampling port defined in the gas collection chamber.

5. The apparatus of claim 4 wherein the sampling port is positioned at or proximate the upper side of the gas collection chamber.

6. The apparatus of claim 1, wherein the intake line and the at least one restrictor line are one in the same.

7. The apparatus of claim 4, further comprising a sample collector which includes:
an interior with adjustable size, and
a mouth removably couplable to the sampling port, where the is removably coupled to the sampling port and the interior of the sample collector is expandable to withdraw at least some of the contents of the gas collection chamber.

8. The apparatus of claim 4, wherein the sampling port comprises a three-position valve having selectable functions of water-out, off, gas-out.

9. The apparatus of claim 8, further comprising an in situ, real-time, gas analyzing detector selectively in fluid communication with the gas collection chamber.

10. The apparatus of claim 1, wherein the at least one treatment station comprises an inlet mixing T upstream of the pump, the inlet mixing T adapted for introducing a treatment fluid into the fluid flow through the apparatus.

11. The apparatus of claim 1, wherein the at least one treatment station comprises an electromagnetic radiation source positioned upstream of the pump, the electromagnetic radiation source adapted to expose fluid flow through the apparatus to electromagnetic radiation.

12. The apparatus of claim 11, wherein the electromagnetic radiation source comprises an ultraviolet light source.

13. The apparatus of claim 1, wherein the at least one treatment station comprises at least one inlet mixing T upstream of the pump, the at least one inlet mixing T adapted to introduce a treatment fluid into the fluid flow through the apparatus, and an electromagnetic radiation source positioned upstream of the pump and downstream of the at least one inlet mixing T, the electromagnetic radiation source adapted to expose fluid flow through the apparatus to electromagnetic radiation.

14. The apparatus of claim 1, wherein the restrictor line flow areas of all restrictor lines collectively define a total restrictor flow area which is less than the intake line flow area.

15. The apparatus of claim 1, wherein the pump is removable couplable to a separate driver, whereby the driver is operably coupled to the pump to actuate the pump.

16. The apparatus of claim 15, wherein the pump has a protruding shaft, and wherein a rotary input supplied to the shaft actuates the pump.

17. The apparatus of claim 15, wherein the pump is a gear pump magnetically coupled to a rotary power source.

18. The apparatus of claim 1, wherein at least one of
a. the intake line, and
b. the at least one restrictor line, are flexible.

19. The apparatus of claim 1, wherein at least one of
a. the intake line,
b. the at least one restrictor line, and
c. the collection chamber, are transparent.

20. An apparatus for collecting dissolved gases from groundwater comprising:
an intake line having an inlet end and a downstream outlet end, the intake line having an intake line flow area defined therein;
pumping means for supplying groundwater to the intake line;
restrictor means located upstream of the pumping means and downstream from the intake line, and receiving groundwater flowing therefrom, the restrictor means decreasing the pressure of the groundwater to a degree sufficient that at least a portion of any gases dissolved within the groundwater precipitate therefrom;
at least one treatment station positioned upstream of the restrictor means. the at least one treatment station adapted for selected treatment of groundwater flow through the apparatus;
a gas collection means for collecting at least a portion of any precipitated gases; and
a disposal line leading from the gas collection means, whereby the groundwater from which the gases have been precipitated is disposable from the gas collection means.

21. The apparatus of claim 20, wherein the restrictor means includes at least one restrictor line, extending between an restrictor line inlet end and a downstream restrictor line outlet end and having a restrictor line flow area defined therein, wherein the restrictor line inlet end of the at least one restrictor line is downstream from the outlet end of the intake line.

22. The apparatus of claim 21, wherein the restrictor line flow areas of all restrictor lines collectively define a total restrictor flow area which is less than the intake line flow area.

23. The apparatus of claim 20, wherein the pumping means is located downstream from the restrictor means, and the at least one treatment station is located upstream from the restrictor means.

24. The apparatus of claim 20, wherein the gas collection means includes a gas collection chamber having an upper side, an opposing lower side, and an interior, and wherein the disposal line exits the gas collection chamber at or near the lower side of the gas collection chamber.

25. The apparatus of claim 20, wherein the intake line and the at least one restrictor line are one in the same.

26. The apparatus of claim 20, wherein the at least one treatment station positioned upstream of the at least one restrictor line is selected from the group consisting of at least one inlet mixing T adapted to introduce a treatment fluid into the fluid flow through the apparatus, and an electromagnetic radiation source downstream of the at least one mixing T, the electromagnetic radiation source adapted to expose fluid flow through the apparatus to electromagnetic radiation.

27. The apparatus of claim 24, further comprising a sampling port positioned at or proximate to the upper side of the gas collection chamber.

28. The apparatus of claim 27, further comprising a sample collector which includes:
an interior with adjustable size, and
a mouth removably couplable to the sampling port, whereby the mouth is operably coupled to the sampling port and the interior of the sample collector is expandable to withdraw at least some of the contents of the gas collection chamber.

29. The apparatus of claim 27, wherein the sampling port comprises a three-position valve having selectable functions of water-out, off, gas-out.

30. The apparatus of claim 29, further comprising an in situ, real-time, gas analyzing detector selectively in fluid communication with the gas collection chamber.

31. A process for collecting dissolved gases from groundwater comprising the steps:
supplying groundwater to an intake line having an inlet end and a downstream outlet end;
treating groundwater within the intake line to selectively alter the concentration of any gases dissolve therein;
directing the groundwater through a restrictor line downstream from the inlet end of the intake line;
decreasing the pressure of the groundwater within the restrictor line to a degree sufficient that at least a portion of any gases dissolved within the groundwater precipitate therefrom;
collecting at least a portion of any precipitated gases; and
disposing of the groundwater from which the gases have been precipitated.

32. The process of claim 31, further including the step:
analyzing continuously the at least a portion of any precipitated gases.

33. The process of claim 31, wherein the following steps are performed prior to supplying groundwater to the intake line:
transporting the intake line in a folded state to a groundwater sampling site;
unfolding the intake line at the groundwater sampling site; and
placing the inlet end of the intake line into the groundwater to be supplied to the intake line.

34. The process of claim 31, wherein any precipitated gases are collected in a gas collection chamber, and wherein the gas collection chamber is transported to the groundwater sampling site with the intake line.

35. The process of claim 31, wherein the restrictor line includes at least two restrictor lines.

36. The process of claim 31, wherein the intake line and the restrictor line are one in the same.
